(19) United States
(12) Patent Application Publication
(10) Pub. No.: US 2026/0128149 A1
(43) Pub. Date: May 7, 2026

(54) SEMAGLUTIDE DOSAGE MANAGEMENT

(71) Applicant: Helix, Inc., San Mateo, CA (US)

(72) Inventors: Matthew Levy, Washington, DC (US); Elizabeth Cirulli Rogers, Lakeside, CA (US)

(21) Appl. No.: 19/002,020

(22) Filed: Dec. 26, 2024

Related U.S. Application Data
(60) Provisional application No. 63/717,784, filed on Nov. 7, 2024.

(51) Int. Cl. G16H 20/17; G16H 10/60

(52) U.S. Cl. CPC G16H 20/17; G16H 10/60

(57) ABSTRACT

Systems and methods for determining effectiveness of semaglutide for a patient. One embodiment is a system for analyzing dosage effectiveness. The system is configured to receive health data for a population of patients, extract metrics for sex, sleep apnea, hypertension, and prescription history from the health data, and train a predictive model based on the metrics. The system is configured to identify a patient and a semaglutide dosage, and operate the predictive model to predict a likelihood of the semaglutide dosage accomplishing a selected amount of weight loss for the patient during a time period. In an event the likelihood is below a threshold, the system is configured to recommend that the dosage not be prescribed to the patient. In an event the likelihood is above the threshold, the system is configured to recommend that the dosage be prescribed to the patient.

### RELATED APPLICATIONS

This non-provisional application claims priority to U.S. provisional application 63/717,784, filed on Nov. 7, 2024, which is incorporated herein by reference as if fully provided herein.

### FIELD

The disclosure relates to the field of health care, and in particular to controlling a dosage of semaglutide administered to patients.

### BACKGROUND

Semaglutide is a widely used pharmaceutical that has multiple applications, including for weight control. However, it is not uncommon for semaglutide to be ineffective in driving weight loss for certain patients, or for patients to discontinue semaglutide use due to undesirable side effects, such as bloating or nausea.

Healthcare providers therefore continue to seek out new, robust solutions that enhance the ability to provide semaglutide to patients within a population in an efficacious manner.

### SUMMARY

Embodiments described herein utilize predictive models trained on specific metrics of population data to anticipate the effectiveness of semaglutide dosages upon specific patients. This results in insights which may be used to determine whether to initiate, adjust, or discontinue a default semaglutide dosage for a patient.

One embodiment is a system for analyzing dosage effectiveness. The system includes an interface configured to receive health data for a population of patients, and a controller configured to extract metrics for sex, sleep apnea, hypertension, and prescription history from the health data, and to train a predictive model based on the metrics. The controller is further configured to identify a patient and a semaglutide dosage, and to operate the predictive model to predict a likelihood of the semaglutide dosage accomplishing a selected amount of weight loss for the patient during a time period. In an event the likelihood for the semaglutide dosage is below a threshold, the controller is configured to recommend that the semaglutide dosage not be prescribed to the patient. In an event the likelihood for the semaglutide dosage is above the threshold, the controller is configured to recommend that the semaglutide dosage be prescribed to the patient.

A further embodiment is a method for analyzing dosage effectiveness. The method includes receiving health data for a population of patients, extracting metrics for sex, sleep apnea, hypertension, and prescription history from the health data, and training a predictive model based on the metrics. The method further includes identifying a patient and a semaglutide dosage, and operating the predictive model to predict a likelihood of the semaglutide dosage accomplishing a selected amount of weight loss for the patient during a time period. In an event the likelihood for the semaglutide dosage is below a threshold, the method includes recommending that the semaglutide dosage not be prescribed to the patient. In an event the likelihood for the semaglutide dosage is above the threshold, the method includes recommending that the semaglutide dosage be prescribed to the patient.

A further embodiment is a non-transitory computer readable medium embodying programmed instructions which, when executed by a processor, are operable for performing a method for analyzing dosage effectiveness. The method includes receiving health data for a population of patients, extracting metrics for sex, sleep apnea, hypertension, and prescription history from the health data, and training a predictive model based on the metrics. The method further includes identifying a patient and a semaglutide dosage, and operating the predictive model to predict a likelihood of the semaglutide dosage accomplishing a selected amount of weight loss for the patient during a time period. In an event the likelihood for the semaglutide dosage is below a threshold, the method includes recommending that the semaglutide dosage not be prescribed to the patient. In an event the likelihood for the semaglutide dosage is above the threshold, the method includes recommending that the semaglutide dosage be prescribed to the patient.

A further embodiment is a method for administering semaglutide. The method includes identifying a patient, selecting a dosage of semaglutide for the patient, and operating a predictive model trained upon health data for a population using metrics of sex, sleep apnea, hypertension, and prescription history to predict a likelihood of the dosage accomplishing a loss of at least ten percent body mass for the patient during a time period of one year. In an event the likelihood is below a threshold, the method includes preventing administration of the dosage to the patient. In an event the likelihood is above the threshold, the method includes administering the dosage to the patient.

Other illustrative embodiments (e.g., methods and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a diagram depicting a sample processing architecture in an illustrative embodiment.

FIG. 2 is a block diagram illustrating a health reporting architecture in an illustrative embodiment.

FIG. 3A is a flowchart depicting a method for dynamically controlling dosage for semaglutide based on predicted effectiveness using population metrics in an illustrative embodiment.

FIG. 3B illustrates administration of a semaglutide in an illustrative embodiment.

FIG. 4A is a flowchart depicting a method for identifying efficacious lower dosages of semaglutide using population metrics in an illustrative embodiment.

FIG. 4B illustrates administration of a semaglutide in an illustrative embodiment.

FIG. 5 is a flowchart depicting a method for selecting demographic-specific models for predicting semaglutide effectiveness in an illustrative embodiment.

FIG. 6 is a flowchart depicting a method selectively administering semaglutide to a patient in an illustrative embodiment.

FIG. 7 is a table that summarizes sequencing data for patients and is maintained at a health server in an illustrative embodiment.

FIG. 8 is a table that summarizes variant data for patients and is maintained at a health server in an illustrative embodiment.

FIG. 9 is a table that summarizes biomarker test data for patients and is maintained at a health server in an illustrative embodiment.

FIGS. 10-11 depict Graphical User Interfaces (GUIs) that facilitate ex-ante estimates of semaglutide effectiveness for patients in illustrative embodiments.

FIG. 12 depicts an illustrative computing system operable to execute programmed instructions embodied on a computer readable medium.

### DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

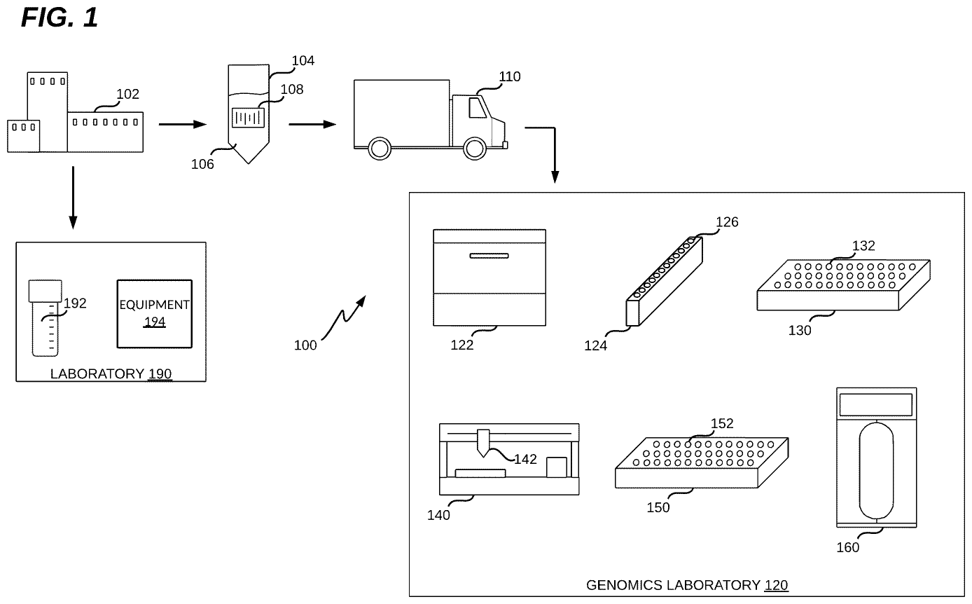

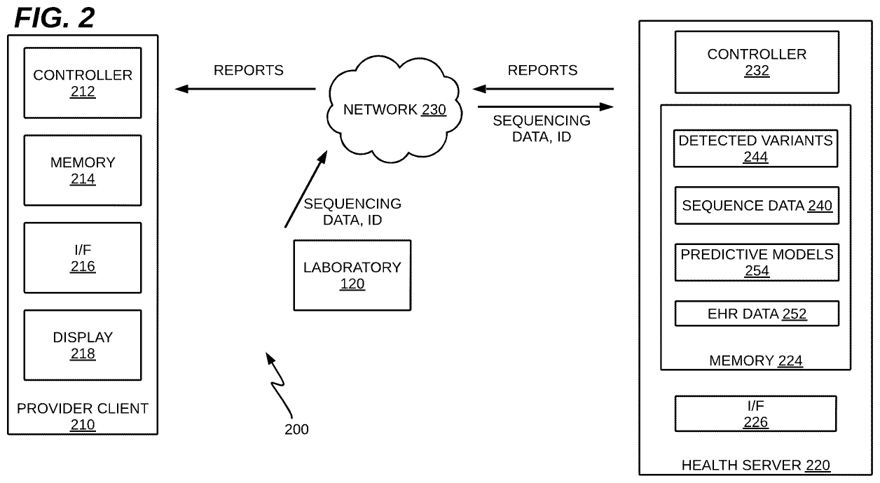

FIGS. 1-2 illustrate illustrative architectures and environments that may interact with methods and systems for predicting semaglutide effectiveness. In particular, FIG. 1 depicts a sampling pipeline via which biological samples may be sequenced, feeding in sequencing data for use in the calculation of population metrics in certain embodiments.

FIG. 1 is a diagram depicting a sample processing architecture 100 in an illustrative embodiment. Sample processing architecture 100 comprises any system or organizational structure for acquiring and sequencing biological samples in a high-volume, high-throughput manner. Sample processing architecture 100 may be utilized, for example, to collect and sequence genetic material (in the form of Deoxyribonucleic Acid (DNA) or Ribonucleic Acid (RNA)) found within thousands or tens of thousands of samples 106 daily, via multiple healthcare provider networks 102.

Healthcare provider networks 102 may comprise hospitals, clinics, practitioner offices, laboratories, surgical centers, etc., that engage in or facilitate the practice of medicine. In one embodiment, healthcare provider networks 102 each comprise groups of hospitals that treat millions of patients. As a part of the practice of medicine, healthcare provider networks 102 acquire samples 106 for sequencing. For example, a healthcare provider network 102 may acquire samples 106 as part of a population screening program, as part of medical treatment, etc. In further embodiments, patients within a healthcare provider network 102 receive sampling kits for independent, self-directed use in acquiring samples 106. The specific amount of sequencing desired for a sample 106 may comprise a selected set of one or more genes, an exome, the entire genome of a patient, etc. The samples 106 are stored in sample containers 104, which may be accompanied by Customer Sample Identifiers (CSIs) 108. A delivery service 110 provides the samples 106 to a genomics laboratory 120 for processing.

Healthcare provider networks 102 may also acquire samples 192 for conventional blood testing (described below). These samples 192 may be provided to laboratory 190 for analysis via equipment 194 (e.g., a chemically treated test strip, biochemical assay, etc.), or may be analyzed by patients via at-home testing methods. For example, a patient may utilize an at-home device to measure blood sugar levels, which are then collected as health record data for the patient. Sample processing architecture 100 provides a technical benefit by allowing laboratory 190 and genomics laboratory 120 to specialize in different methods of analysis.

Procedures within genomics laboratory 120 related to genetics may include accessioning, sample plating, storage, extraction, library preparation, enrichment, and sequencing processes. These processes acquire genetic material from a sample 106, separate the genetic material from other constituents, duplicate the genetic material, and quantify the genetic material order to determine a swathe of sequence data, such as an exome or entire genome for a subject (e.g., a human patient, an organelle of a human patient, etc.). Although the procedures discussed herein are specific with regard to one method of sequencing, other techniques may be utilized in accordance with known standards in order to perform sequencing for samples 106. For example, although certain short-read technologies herein are discussed as utilizing hybridization capture techniques, amplicon-based techniques may be used alternatively or to supplement those techniques. Long-read techniques may also or alternatively be utilized.

### Accessioning

Accessioning refers to receiving and preparing samples 106 for later laboratory processes. In one embodiment, accessioning includes receiving a batch of samples 106 (e.g., hundreds or thousands of samples 106) from one or more delivery services 110 each day for processing. For example, packages that each include tens or hundreds of samples 106 may be delivered to genomics laboratory 120 via the United States Postal Service (USPS), or a private package carrier.

Each sample 106 may be retained within a sample container 104, such as a five milliliter (mL) test tube. In this embodiment, the sample container 104 is sealed to prevent the sample 106 from being exposed to the environment and also to prevent the sample 106 from co-mingling with other samples 106. For example, the sample 106 may be sealed via a cap that is threaded, glued, press-fit, etc. At the time of delivery, the sample container 104 may further include a remnant of a sampling tool, such as a portion of a swab that was utilized to acquire the sample.

In many embodiments, a CSI 108 for the sample 106 is reported via a component affixed to or integrated with the sample container 104. The CSI 108 uniquely distinguishes the sample 106 from other samples 106 being received. For example, a CSI 108 may uniquely distinguish a sample 106 from other samples 106 in the same batch, other samples 106 received on the same date, other samples 106 received from the same healthcare provider network 102, etc. A CSI 108 may be reported via a barcode label, Quick Response (QR) code label, Radio Frequency Identifier (RFID) chip, or any suitable visual, transmission-generating, or other physical component affixed to or integrated with the sample container 104.

In further embodiments, the sample container 104 is itself sealed within an external container such as a bag (not shown). Using an external container helps to prevent contamination, by ensuring that a technician at the genomics laboratory 120 does not contact biological material from the sample 106 that may exist on an outer surface of the sample container 104. Use of an external container may also be required by law (e.g., Department of Transportation (DOT) guidelines). Use of an external container additionally helps to prevent cross-contamination between samples 106. Furthermore, in embodiments where samples 106 may include blood or a pathogen, an external container provides an additional barrier to protect the health of technicians. The external container may additionally include documentation confirming the CSI 108, information for the subject that the sample was sourced from, and/or information indicating circumstances of sampling. The circumstances of sampling may include, for example, a sampling date, a sampling method, a location that the sample was acquired, a name or title for a person who performed the sampling, and/or additional notes.

In this embodiment, the sample 106 comprises a chemical solution. For example, the sample 106 may comprise a prepared aqueous solution such as a saline solution, or may comprise a bodily fluid such as blood, saliva, mucus, etc. In some embodiments, each of the samples 106 fills between two and five milliliters of volume within its corresponding sample container 104. In further embodiments, the samples 106 may be constituted at the genomics laboratory 120 from dried blood spots applied to filter paper, may comprise buccal material, etc.

The samples 106 further include genetic material such as Deoxyribonucleic Acid (DNA), Ribonucleic Acid (RNA), etc. In many instances, the genetic material is one of many constituent components within the sample 106. For example, the genetic material may exist within the nuclei of white blood cells that are included within the sample 106. In a further example, genetic material may exist within viruses or bacteria within the sample 106. In this embodiment, the genetic material is not yet isolated from the remaining constituent components of the sample 106.

After receipt of the samples 106, batches of the samples 106 (e.g., as stored within sample containers 104 and/or external containers) may be heated in ovens 122 to facilitate cell lysis. The temperature, and duration of heating, may be chosen such that pathogenic material within the samples 106 is rendered harmless, or such that cellular lysis occurs. For example, heating may occur at a temperature in a range of about forty and eighty (e.g., fifty) degrees Celsius (C), for a period of time in a range of about fifteen and two hundred (e.g., thirty) minutes. In some embodiments, including embodiments where the samples 106 are primarily the contents of a blood draw, the heating step may be foregone.

In this embodiment, upon completion of heating, the batches of samples 106 are removed from the ovens 122. In one embodiment, sample containers 104 are removed from corresponding external containers, such as by cutting the external containers open. With the sample containers 104 now available for direct interaction, the sample containers 104 are inspected. As a part of this process, a technician or automated system may determine the CSI 108 for the sample 106, and may compare the CSI 108 to a CSI 108 listed on documentation provided in the external container. If there is a discrepancy between the CSI 108 on the sample container 104 and a CSI 108 listed in the documentation, the sample 106 may be flagged as having an error condition. Similarly, if the CSI 108 on the sample container 104 is damaged (e.g., abraded, heat-damaged, or water-damaged) and has become unreadable, the sample 106 may be flagged as having an error condition.

A technician or automated system may further inspect the contents of the sample container 104, via visual or other methods. If the sample 106 does not include an expected constituent component (or is otherwise non-compliant), then the sample 106 is flagged as having an error condition. For example, if the sample 106 is primarily saliva and includes a fluid that is not permitted (e.g., blood), includes an entire swab or no swab, appears to have a fractured or broken casing, or is outside of an expected range of volume (e.g., between two and five milliliters), then the sample 106 may be flagged as having an error condition.

Samples 106 that have not been flagged as having an error condition proceed to sample integration. In one embodiment, as a part of sample integration, the sample 106 is assigned a Laboratory Sample Identifier (LSI). The LSI uniquely identifies the sample 106 from other samples 106 received for the batch, received on the same day, processed in the same laboratory, and/or handled by the same organization performing sequencing. In many embodiments, the LSI is stored in a memory of a genomics server (e.g., within a laboratory sample database), and is uniquely associated with a corresponding CSI 108 for the sample. The LSI may also be associated with any error conditions reported for the sample 106.

In many embodiments, CSIs 108 originally provided with the samples 106 are in the form of a paper barcode. In such embodiments, the paper barcode may be printed in aqueous ink. This renders the barcode subject to degradation upon exposure to liquid in the laboratory environment, which is undesirable.

To ensure that each sample container 104 is capable of traveling through the genomics laboratory 120 without its identifier being physically degraded, a corresponding LSI may be indicated at the sample container 104. The LSI may be indicated via the application of a barcode label, Quick Response (QR) code, Radio Frequency Identifier (RFID) chip, or other visual, transmission-generating, or other physical component affixed to or integrated with the sample container.

In one embodiment, the LSI is printed onto a barcode label comprising rip-proof material (e.g., vinyl) in a water-insoluble ink. This implementation ensures that the barcode label is resistant to physical and chemical degradation. The barcode may be applied around an entire perimeter of the sample container 104, ensuring that the sample container 104 may be scanned from any angle.

In further embodiments, the element used to report the LSI is accompanied by a visually distinct mark that enables rapid confirmation by a technician that the sample 106 has been integrated into the laboratory environment. The visually distinct mark may comprise a colored ring (e.g., around an entire perimeter of the sample container), a logo, a physical feature, a stamp, etc.

### Sample Plating

With the samples 106 having been successfully integrated into the environment of the genomics laboratory 120, the samples 106 are ready for analytics to be performed. To this end, the samples 106 are prepared for transfer to a sample microplate 130. The sample microplate 130 may be labeled with a unique identifier via similar techniques to those used for sample containers 104 above. The unique identifier distinguishes the sample microplate 130 from other sample microplates 130. In one embodiment, the sample microplate 130 comprises a solid body defining three hundred and eighty-four wells, distributed across sixteen rows and twenty-four columns, each well having a capacity of between thirty and one hundred microliters. In a further embodiment, the sample microplate 130 comprises a solid body defining ninety-six wells, distributed across eight rows and twelve columns, each well having a capacity of between one hundred and three hundred microliters. Any suitable number and arrangement of wells may be selected as a matter of design choice.

As a part of preparing the samples 106 for transfer to the sample microplate 130, a technician may place sample containers 104 onto a rack 124, and scan each sample container 104 to determine an LSI for each location 126 (e.g., each container receptacle) on the rack 124. In some embodiments, the rack 124 is assigned a unique identifier that distinguishes it from other racks 124. The rack 124 may be labeled with a unique identifier using techniques similar to those used for sample containers 104. The technician, or automated machinery such as a server operating an optical scanner, may then associate the unique identifier for the rack 124, along with the locations 126 assigned to the samples 106, with the corresponding LSIs of the samples 106 stored at the rack 124.

The technician additionally unseals the sample containers 104. Unsealing of sample containers 104 may be a deeply labor-intensive process, particularly when laboratory processes are performed at scale to handle tens of thousands of samples 106 per day. Thus, a technician may utilize automated tooling to enhance the speed at which sample containers 104 are unsealed. The tooling may, for example, lever open, pull, unscrew, cut, or drill each sample container 104, in order to make the sample 106 within available for physical transfer to the sample microplate 130.

One or more racks 124 of samples 106 are provided to a Liquid Handler (LH) 140, such as an automated robot that operates an end effector 142 in accordance with one or more Numerical Control (NC) programs to transfer liquids between wells via arrays of micropipettes. An LH 140 is also known as a “Liquid Handling System”. LH 140 may comprise, for example, a Hamilton Microlab Star Liquid Handling System.

In this embodiment, the LH 140 proceeds to transfer a portion of each sample 106 at a rack 124 to a well 132 within the sample microplate 130. The well 132 is not shared with (i.e., is distinct from) wells 132 for other samples 106. For example, the well 132 for each sample 106 may be predetermined in accordance with a control program used by the genomics laboratory 120. In one embodiment, the LH 140 transfers the portions of the samples 106 to the wells 132 of the sample microplate 130 by providing instructions to actuators, piezoelectric elements, and/or pressure systems operating the end effector 142. In such an embodiment, the end effector 142 may align its array of micropipettes with the sample containers 104 to retrieve portions of the samples 106. Furthermore, in such an embodiment, the end effector 142 may dynamically align its array of micropipettes with the sample microplate 130 to deposit the portions of the samples 106 at the wells 132.

Because there is a known relationship between locations 126 at the rack 124 and wells 132 of the sample microplate 130 (e.g., as indicated by row and column), contents of the memory of a genomics server (e.g., a laboratory sample database) may be updated to indicate the well 132 storing genetic material for each sample 106. In one embodiment, the memory is further updated to associate a unique identifier for the sample microplate 130 with the samples 106 stored therein.

In one embodiment, programmed instructions for the LH 140 may direct the end effector 142 to position itself above a set of disposable tips, descend into the tips to attach the tips, reposition the end effector 142 above the rack of sample containers 104, adjust spacing between micropipettes within the array, descend until the tips reach the sample containers 104, draw liquid from the sample containers 104, deposit the liquid into a well 132 at the sample microplate 130, and then dispose of the tips. Such a process may be repeated across sample containers 104 stored on multiple racks until the sample microplate 130 is filled with portions from the samples 106. In one embodiment, one or more wells 132 on the sample microplate 130 are filled with a control reagent instead of a portion of a sample 106.

The amount of liquid drawn from each sample container 104 may comprise a small fraction of the overall volume of the sample container 104. For example, an amount of liquid drawn may comprise several microliters, such as between two and ten microliters. Upon completion of transfer from the sample containers 104 to the wells, the sample microplate 130 may be covered with a liquid and/or gas-impermeable layer, such as foil or paraffin. Sample containers 104 remaining on the racks may be resealed, for example with pressure-fit caps having a color distinct from an original color for the sample containers. With accessioning now complete for the sample microplate 130, the sample microplate 130 is transferred to a next section of the laboratory for processing.

In embodiments where the genomics laboratory 120 performs both short-read and long-read sequencing workflows, the sample plating techniques discussed above may be performed separately, asynchronously, and/or in parallel for short-read technologies (e.g., via an Illumina sequencing platform such as a NovaSeq X) and for long-read technologies (e.g., via a PacBio sequencing platform such as a Revio). These techniques may also vary between long-read sequencing workflows and short-read sequencing workflows. For example, the number and nature of plates used for samples 106, the amount of sample 106 used for the sequencing workflow, and whether a process is manual or automated all may vary between sequencing workflows. For example, these differences may occur in the workflows to support the requirements of different pieces of sequencing equipment 160, to account for differences in sequencing volume between workflows, etc. Samples 106 received at the genomics laboratory 120 may include sufficient genetic material to support multiple sequencing processes (e.g., both short-read and long-read sequencing processes). Thus, in many embodiments, samples 106 provide genetic material for both short-read and long-read sequencing, supporting the rigor of diagnostic genetic testing processes.

### Storage

In one embodiment, accessioned samples 106, samples 106 ready for analytics, and/or samples 106 that have already been sequenced, are stored for later use. For example, samples 106, sample containers 104, and/or sample microplates 130 may be stored at room temperature, or may be cryogenically frozen at a low temperature (e.g., negative eighty degrees Celsius) within a freezer and arranged in racks for later retrieval. Samples 106 may be preserved for periods of days or years, enabling rapid re-testing to be performed for subjects without the need for re-acquiring genetic material. Storage of the samples 106 provides notable value in the event that contents of a well 132 used for sequencing do not meet with rigorous quality control standards. Specifically, storage enables re-sampling to occur in the event that there is a desire to re-sequence a sample 106.

### Extraction

Sample microplates 130 are transferred to a portion of the genomics laboratory 120 dedicated to extraction of the genetic material. The segment of the laboratory 120 that performs extraction and other pre-amplification operations may be scaled from, and/or positively pressurized relative to, other portions of the genomics laboratory 120.

During extraction, a sample microplate 130 is acquired and provided to an LH 140. The LH 140 that performs extraction may be different from the LH 140 that performs sample plating. The LH 140 may apply a reagent to each well 132 that lyses cells within each well. For example, this may be performed in order to lyse white blood cells containing genetic material for a human, or may comprise lysing other types of cells or components to expose other types of genetic material. The reagents used for pre-amplification processes may be stored at the LH 140 in a temperature-controlled manner, and may even be vibrated or mixed on a regular basis to ensure that the reagents are evenly distributed in suspension.

In one embodiment, extraction further includes an LH 140 aspirating and dispensing reagents that selectively bind to genetic material released from the lysed cells. This process may include applying a bead (not shown) to the well 132. In one embodiment, the beads comprise magnetic beads that selectively bind to the genetic material (e.g., DNA). This allows for isolation and purification of the genetic material while contaminants remain in solution. In one embodiment, the magnetic bead is drawn to a magnetic base at or under the sample microplate 130. After the genetic material has been drawn to the bead, and after the bead has been secured to the base of the well, a flushing step may be performed where remaining fluid in each well is washed away. This ensures that potential impurities are removed from the well 132. The LH 140 may further add or remove fluid from each well 132 to perform additional concentration and/or elution of the genetic material, and may transfer fluid from the wells 132 of the sample microplate 130 to wells 152 of a genome stock microplate 150. The genome stock microplate 150 may be labeled with a unique identifier, and the contents of each well 152 of the genome stock microplate 150 may be associated with a corresponding LSI. In all phases of operation, the LH 140 is operated to ensure that fluid is not transferred between wells 152, as this results in contamination by intermingling genetic material for different samples 106.

In one embodiment, a portion of fluid is removed from each well 152 of the genome stock microplate 150 for quality control purposes. Concentration of genetic material within the wells 152 may be confirmed via testing of this fluid, such as by application of a dye that reacts with the genetic material at known levels of fluorescence for known concentrations.

In embodiments where the genomics laboratory 120 performs both short-read and long-read sequencing workflows, the extraction techniques discussed above may be performed separately, asynchronously, and/or in parallel for short-read technologies (e.g., via an Illumina sequencing platform such as a NovaSeq X) and for long-read technologies (e.g., via a PacBio sequencing platform such as a Revio).

### Library Preparation

After extraction is completed, library preparation may be performed for the contents of the genome stock microplate 150. The bead for each well 152, including ionically bonded genetic material, is transferred to a distinct well of a library preparation microplate (not shown). The library preparation microplate includes an identifier that uniquely distinguishes it from other library preparation microplates, and the LSI associated with each well 152 on the genome stock microplate 150 may be mapped to a corresponding well on the library preparation microplate.

The library preparation microplate may be transferred to a new portion of the genomics laboratory 120 that is sealed from, and/or positively pressurized relative to, other portions of the genomics laboratory 120 that do not perform amplification of genetic material. This feature helps to prevent amplified genetic material from entering portions of the laboratory where genetic material has not been amplified, which could result in contamination. The transfer process may be performed by placing a library preparation microplate into an airlock at the pre-amplification portion of the genomics laboratory 120, scaling the airlock, and then retrieving the library preparation microplate from the airlock via the amplification portion of the genomics laboratory 120.

In one embodiment, a reagent is applied to each well of the library preparation microplate. The reagent ionically bonds to the surface of the bead within the well, and does so more strongly than the genetic material. This releases the genetic material from the surface of the bead of each well, enabling the genetic material to be chemically interacted with.

Library preparation may include normalization of a concentration of genetic material in each well of the library preparation microplate. Library preparation further includes fragmentation of the genetic material via an enzyme or via the application of physical forces. During this process, the entire genome (e.g., roughly three billion base pairs for a human genome), may be fragmented into pieces. In one embodiment where short-read sequencing is performed, the pieces vary between three hundred and four hundred base pairs in length. These pieces are known as nucleic acid fragments. In a further embodiment where long-read sequencing is performed, the pieces may vary between five hundred and fifty thousand or more base pairs in length.

In one embodiment utilizing short-read sequencing, the nucleic acid fragments undergo adaptor ligation and indexing in accordance with known techniques. For example, this may comprise Next Generation Sequencing (NGS) library preparation processes defined by Illumina. Next, a limited amount of Polymerase Chain Reaction (PCR) amplification is performed upon the library. The resulting solution is then purified and eluted via operation of an LH 140.

During library preparation, one or more reference samples of genetic material, distinct from the genetic material found in the samples, may be added to wells of the library preparation microplate. The reference samples do not include genetic material received from a customer, but rather include known sequences of base pairs. The reference samples serve as controls to ensure that processes are carried out with sufficient quality.

Upon completion of library preparation, desired fragments of the genetic material (e.g., thousands or millions of distinct fragments of the genetic material, each corresponding with a different portion of a genome of the subject) have been ligated to predefined adapters (e.g., DNA adapters) that bind with the genetic material. Each of the adaptor-ligated fragments is referred to as a “library”.

In further embodiments, the probes applied to each well of the library preparation plate include chemical identifiers (colloquially referred to as “barcodes”) that are distinct from each other. The use of a different chemical identifier for probes applied to each well of the library preparation microplate enables sequencing to later be performed for multiple subjects on the same flow cell, without conflating sequencing results for those subjects.

In one embodiment utilizing long-read sequencing, library preparation may be performed via physical shearing of DNA to achieve a target size distribution mode between ten and twenty-five kilobases (kb), such as between fifteen and eighteen kb. The resulting nucleic acid fragments may be coupled to adapters to prepare them for sequencing via Single-Molecule Sequencing in Real Time (SMRT) or other long-read technologies.

The library preparation processes discussed herein may further comprise controlling a concentration of the genetic material in each well, and purification and/or elution of the resulting material. Similar to the processes performed after extraction of genetic material, concentration of genetic material after library preparation may be confirmed for each well via testing.

### Enrichment

After library preparation, enrichment processes may be performed in order to either directly amplify (e.g., via amplicon or multiplexed PCR) or capture (e.g., via hybrid capture) predefined libraries. This enhances the case of sequencing desired portions of the genome. In some embodiments, enrichment is foregone for long-read sequencing processes.

In one embodiment, during enrichment, customized biotinylated oligonucleotide probes are applied to the libraries. The probes selectively hybridize genetic material occupying desired portions of the genome for the genetic material, such as specific genes, or the entire exome. Magnetic beads bind to biotin molecules in the probes to attach the hybridized material to the magnetic beads. Magnetic forces capture the beads in place, enabling remaining fluid within each well to be removed or washed out, thereby removing impurities and leaving only the genetic material that is desired. Genetic material may be released from the beads in a similar manner to that discussed above for prior processes.

In a further embodiment, hybrid capture target enrichment is performed. During this process, the probes comprise tailored oligonucleotides that are chosen to bind to the genetic material. The range of probes may be tailored as a group to bind to specific alleles, specific genes, the exome, the entire genome, etc. That is, each probe may bind to a nucleic acid fragment at a specific location on the genome, and the range of probes may be selected to ensure that alleles, genes, the exome, or the entire genome of the subject being considered is acquired. Utilizing probes in this manner may enhance efficiency of the sequencing process, by foregoing sequencing of all of the roughly three billion base pairs found in the human genome.

The enrichment process may further comprise controlling a concentration of the genetic material in each well, and purification and/or elution of the resulting material. Similar to the processes performed after extraction of genetic material, concentration of genetic material after enrichment may be confirmed for each well via testing.

### Sequencing

Sequencing may be performed according to any of a variety of techniques, including short-read and long-read techniques, via sequencing equipment 160 (e.g., an Illumina NovaSeq X sequencing machine, a PacBio Revio sequencing machine, etc.). As used herein, short-read sequencing refers to sequencing technologies that generate reads of five hundred or fewer base pairs in length. Short-read sequencing may be used as the basis for “synthetic long read” technologies that stitch individual short reads together, but as used herein, short-read sequencing does not refer to the creation or use of synthetic long reads.

In one embodiment, short-read sequencing is performed as Sequencing by Synthesis (SBS). For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers referred to above. In one embodiment, the chemical identifiers comprise nucleotide sequences that are detectable during the sequencing process to determine a corresponding LSI.

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

In this embodiment, sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence that can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Additionally in this embodiment, base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome or another set of genomic data is being tested, different reagents (e.g., probes, primers, etc.) may be chosen. That is, different reagents and/or processes may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

In a further embodiment, long-read sequencing (e.g., sequencing of nucleic acid fragments larger than one kilobase) is performed in an SMRT process, where nucleic acid fragments are circularized and bound to a DNA polymerase enzyme. The bound pair enter a sequencing chamber, and the DNA polymerase adds complementary bases to the DNA strand that are fluorescently labeled to result in different colors for different bases.

As labelled bases are added by the polymerase, the color of the base is recorded, and then the fluorescent label is removed. The next base for the circularized nucleic acid fragment is then added and recorded, iteratively, until the circularized nucleic acid fragment has been sequenced a desired number of times.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

### Bioinformatics

Sequencing data may be stored in any suitable format. In one embodiment, raw sequencing data generated during short-read sequencing is stored in a file format, such as Binary Base Call (BCL). This raw data may be fed to an analytical pipeline, such as a cloud-based computing environment. Raw sequencing data may be processed by the pipeline into a second format, such as a text-based FASTQ format, that reports quality scores. The second format may then be analyzed to perform alignment of sequence reads to a reference genome, such as a reference genome reported in a Browser Extensible Data (BED) file. The aligned sequence data may be reported as a Binary Alignment Map (BAM) file or Compressed Reference-oriented Alignment Map (CRAM) file. In one embodiment, long-read sequencing data is output from the corresponding sequencing machine as one or more BAM files, obviating the need for long-read sequence data undergoing the conversion processes discussed above.

The aligned sequence data may then be called, resulting in a Variant Call Format (VCF) file reporting called variants at each location of the genome that was sequenced, together with secondary metrics, such as quality indicator metrics. As used herein, a variant comprises a unique combination of genetic information, in the form of consecutive base pairs at a specific set of locations (e.g., genomic coordinates) along a portion of a chromosome or other genomic segment. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, insertions and/or deletions (Indels) which relate for example to the insertion or deletion of less than thirty base pairs, or differing numbers of repetitions, Copy Number Variants (CNVs), which relate to larger insertions or deletions, translocations, inversions, other types of genetic variants, or even combinations of variants, such as haplotypes or Multi-nucleotide variants (MNVs).

The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Health Reporting Architecture

FIG. 2 is a block diagram illustrating a health reporting architecture 200 in an illustrative embodiment. Health reporting architecture 200 comprises any combination of systems and devices operable to review, process, and/or control access to health data, such as Electronic Health Record (EHR) data 252 from healthcare providers, and/or sequencing data received from genomics laboratory 120. In this embodiment, health reporting architecture 200 comprises a health server 220 which receives EHR data relating to patients within a population (e.g., hundreds of thousands, or millions, of patients). The EHR 252 data may be received via interface (I/F) 226, such as an Ethernet interface, wireless interface compliant with Institute of Electrical and Electronics Engineers (IEEE) 802.11 standards, or other physical interface capable of transmitting and receiving digital data. Controller 232 may format the EHR data 252 into a standardized format to facilitate analysis of the EHR data 252 as stored in memory 224. For example, the EHR data 252 may comprise records that have been rendered into a uniform format, such as an Observational Medical Outcomes Partnership (OMOP) format, and may comprise health records for each patient that sequencing data has been stored for. In one embodiment, the EHR data 252 includes content coded according to one or more medical vocabularies (e.g., International Classification of Diseases (ICD), Current Procedural Terminology (CPT), OMOP Common Data Model (CDM) vocabularies, and/or others). This arrangement facilitates rapid identification of related concepts.

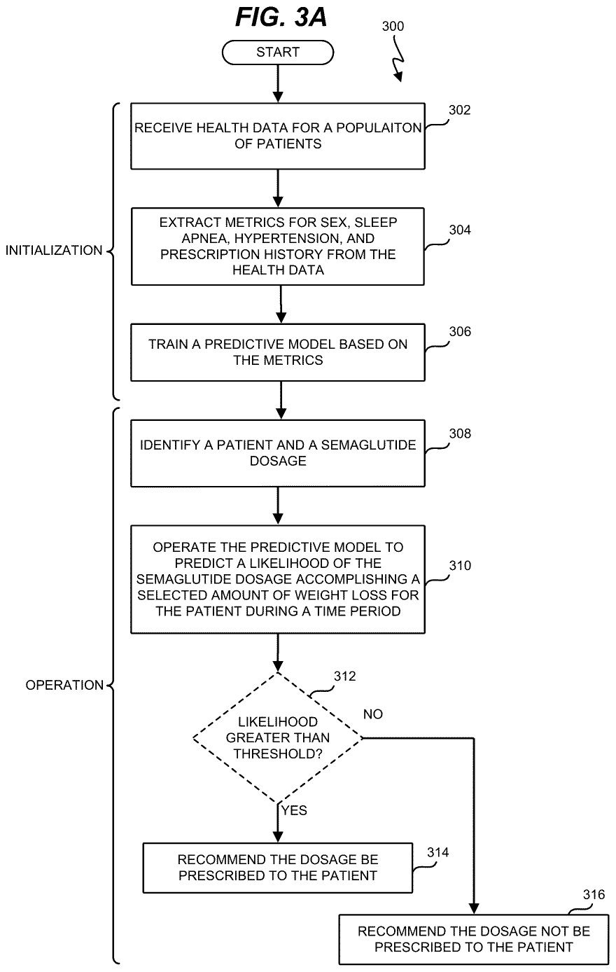

Memory 224 further stores one or more predictive models 254, which comprise analytical models capable of predicting a likelihood that a dosage of semaglutide will effectively reduce a target amount of weight for a patient, based on a combination of health-related metrics for that patient. Predictive models 254 may be targeted to (and/or trained using) specific demographic combinations (e.g., sex, age and sex, sex and ancestry, ancestry, ancestry and sex and age, ancestry and age, etc.), or may be targeted to (and/or trained to predict) specific amounts of weight loss (e.g., fifteen percent of body mass, ten percent of body mass, seven-and-a-half percent of body mass, five percent of body mass, etc.). Predictive models 254 are discussed in further detail below with regard to FIGS. 3-6.

In a further embodiment, health server 220 receives sequencing data (also referred to as sequence data) and identifiers (e.g., CSIs 108, LSIs, etc.) from genomics laboratory 120, via network 230. The sequencing data 240 received and processed by the health server 220 may be supplied for multiple different types of sequencing operations, including short-read and long-read sequencing operations. Thus, after sequencing data 240 for a patient has been acquired, it can be maintained at health server 220 in order to facilitate future studies associating relationships between genetic variants and phenotypes. This means that in such embodiments, health server 220 has readily-available access to clinico-genomic data sets that may be highly desirable for deriving treatment-related insights.

Health server 220 receives the sequencing data 240 via I/F 226. The sequencing data 240 is stored in memory 224 for the population of patients that have been sequenced by laboratory 120, and may be maintained in any suitable format. The population of patients that have been sequenced may comprise the same population of patients for the EHR data 252, or a subset of those patients. Examples of suitable formats for sequencing data 240 include CRAM, VCF, BAM, and others. Memory 224 may store, for example, sequencing data 240 describing multiple patients, and this sequencing data 240 may be maintained in a de-identified format to facilitate the advancement of research. Memory 224 may be implemented via a cloud storage service, or may comprise a storage medium such as a hard disk or flash memory device.

Controller 232 manages the operations of health server 220, and may for example, analyze EHR data 252 and/or sequencing data 240 to determine the expected effectiveness of semaglutide for individual patients. In embodiments where controller 232 reviews sequencing data 240, controller 232 may determine alignments to a reference genome in order to identify detected variants 244. Controller 232 may further control access and authentication related to EHR data 252 and/or sequencing data 240, communicate with one or more provider clients 210, and/or perform additional operations. Controller 232 may be implemented, for example, as custom circuitry, as a hardware processor executing programmed instructions, as a combination of shared hardware processing resources implementing a compute service, or some combination thereof.

In further embodiments where semaglutide effectiveness is not calculated using genomic metrics, the processes discussed herein related to sequencing, storage, and/or analysis of sequencing data may be foregone.

Health reporting architecture 200 further comprises provider client 210, which is configured to permit users to interact with health server 220 in order to gain insights related to semaglutide dosage. In some embodiments, provider client 210 is further configured to facilitate health-related activities, such as control of EHR data available on a network of the healthcare provider.

In the embodiment depicted in FIG. 2, provider client 210 includes a controller 212, a memory 214, an interface (I/F) 216, and a display 218. Controller 212 manages the operations of the provider client 210, and may be implemented, for example, as custom circuitry, as a hardware processor executing programmed instructions, or some combination thereof. Memory 214 comprises information for interpreting the data received via I/F 216. Display 218 may comprise a projector, screen, etc., for presenting information to a user of provider client 210.

### Treatment Based on Semaglutide Effectiveness Analysis

FIG. 3A is a flowchart depicting a method 300 for dynamically controlling dosage for semaglutide based on predicted effectiveness for a patient, using population metrics in an illustrative embodiment. The steps of the flow charts described herein are not all inclusive and may include other steps not shown, and the steps may be performed in an alternative order. For example, steps that are depicted with dashed lines are explicitly provided as optional in both their inclusion and order, although this may apply to other steps as well. By controlling treatment based on predicted effectiveness of semaglutide using a bespoke set of factors having values that vary from patient to patient, health server 220 beneficially derives insights that are specific to that patient, rather than applicable to the general population. FIG. 3B illustrates administration of semaglutide 322 in an illustrative embodiment.

Steps 302-306 of FIG. 3A describe an initialization phase, where a predictive model 254 is trained to anticipate effectiveness for semaglutide in individual patients, based on health metrics reviewed across a population of patients. After the initialization phase has been completed, the steps in the operation phase that follows may be iterated any number of desired times to predict effectiveness for individual patients. Furthermore, the predictive model, once trained, may anticipate semaglutide effectiveness for patients that are not within the set of patients that the predictive model was trained on.

Step 302 comprises controller 232 receiving health data for a population of patients. In one embodiment, this comprises receiving EHR data for patients served by one or more networks of healthcare providers, and standardizing the data to create EHR data 252. In a further embodiment, receiving health data may involve generating surveys for the population of patients, receiving answers to the surveys, and compiling answers to surveys by the patients. Each survey question may inquire about a specific metric relevant to training the predictive model.

Step 304 comprises controller 232 extracting metrics for sex, sleep apnea, hypertension, and prescription history from the health data. In one embodiment, this comprises retrieving fields from EHR data 252 that report the metrics. In embodiments where surveys are provided to patients, this may comprise retrieving and processing survey answers directed to the metrics. In one embodiment, a metric for sex reports sex assigned at birth for individual patients in the population, a metric for sleep apnea categorizes a severity of sleep apnea (if any) for individual patients in the population (e.g., as mild, moderate, or severe), a metric for hypertension indicates a severity of hypertension (if any) for individual patients in the population (e.g., as pre-hypertension, hypertension, etc.), and a metric for prescription history indicates a history of prescriptions previously assigned to the patient, such as concurrent use of a non-Glucagon-like peptide-1 (GLP-1) weight loss drug with semaglutide, and previous use of a GLP-1 weight loss drug within a year immediately prior to prescription of semaglutide. In further embodiments, the metrics further comprise a polygenic score for Body Mass Index (BMI), such as the polygenic score calculated in Tanigawa Y, Qian J, Venkataraman G, et al., “Significant sparse polygenic risk scores across 813 traits in UK Biobank,” PLOS Genet 2022; 18: e1010105. and available in the PGS Catalog as PGS001228, or Lambert S A, Wingfield B, Gibson J T, et al. “Enhancing the Polygenic Score Catalog with tools for score calculation and ancestry normalization.” Nat Genet 2024; published online Sep. 26, 2024. DOI: 10.1038/s41588-024-01937-x.

Step 306 comprises controller 232 training a predictive model 254 based on the metrics. Training may comprise performing logistic regression upon the predictive model 254, using metrics and outcomes for individual patients (e.g., as measured by changes in BMI) as training data. In further embodiments, other training processes may comprise implementing a machine learning algorithm to train the predictive model, based on the metrics and outcomes. Depending on the embodiment, the predictive model 254 may be trained to anticipate a likelihood of achieving a specific amount of weight loss (e.g., as a percentage of body mass), may anticipate a most-likely amount of weight loss for a given prescription strength for semaglutide, etc. In many embodiments, controller 232 trains multiple predictive models 254, such as a separate model to anticipate each possible dosage of semaglutide (e.g., 2.4, 1.7, 1.0, or 0.5 milligrams per week), and/or trains different models for different demographics, and/or different models for predicting a likelihood of achieving different desired/target levels of weight loss.

In many embodiments, semaglutide use may be desired to address diabetes-related conditions of a patient. Hence, weight loss is not a relevant factor in the prescription history for certain patients in the population. To account for potential bias relating to diabetes-related prescriptions, controller 232 may exclude metrics for patients of the population that have type two diabetes, prior to training the predictive model(s) 254.

After one or more predictive models 254 have been trained, the health reporting architecture 200 is prepared to actively engage in anticipating semaglutide effectiveness for one or more patients, such as patients who are seeking to achieve weight loss but have not yet been prescribed semaglutide.

Steps 308-316 discuss an operation phase, involving the use of one or more predictive models 254 that have been trained in order to anticipate semaglutide effectiveness and/or alter treatment for specific patients. Steps 310-316 may be performed massively in parallel and/or asynchronously for multiple patients within the population over time. Furthermore, steps 308-316 do not require a separate initialization phase each time a predictive model 254 is used to analyze semaglutide effectiveness for a patient. Note that while controller 232 is described as performing operations in both the operation phase and the initialization phase, in further embodiments, these phases may be operated by controllers of different servers, such as a health server dedicated for training predictive models, and a health server dedicated to servicing requests from provider clients.

Step 308 comprises controller 232 identifying a patient 320 and a semaglutide dosage 324 of a semaglutide 322 (see FIG. 3B). In one embodiment, this comprises I/F 226 receiving a request from a provider client 210, and identifying a unique ID for a patient 320 that is being considered for a semaglutide prescription. Controller 232 may further retrieve EHR data 252 for the patient 320 to retrieve metrics for the patient 320, or may directly receive the metrics as a part of the request. In some embodiments, metrics relating to genetics, such as a BMI polygenic score, are not included in the request. In such circumstances, controller 232 may dynamically determine such metrics by reference to sequencing data 240.

Controller 232 may assume that the semaglutide dosage 324 is a default dosage for weight loss (e.g., 2.4 mg/wk), or controller 232 may extract a dosage that is explicitly reported in the request. In further embodiments, the default dosage may include an expectation of escalating through 0.25 mg, 0.5 mg, and 1 mg, each for four weeks, before finalizing at 1.7 mg or 2.4 mg. Other, smaller dosages, including off-label dosages such as 2 mg, may also be considered.

Step 310 comprises controller 232 operating the predictive model 254 to predict a likelihood 330 of the semaglutide dosage 324 accomplishing a selected amount of weight loss for the patient 320 during a time period. The selected amount of weight loss may be included in the request, or set to a default amount (e.g., ten percent of body mass) by controller 232. Illustrative amounts of weight loss may comprise five percent, seven-point-five percent, ten percent, twelve percent, fifteen percent, etc., of body mass. Other amounts of weight loss may comprise ten pounds, twenty pounds, thirty pounds, forty pounds, fifty pounds, etc.

In further embodiments, percent weight loss or pounds of weight loss may be undesirable, as these measurements may include biases relating to some body types or to heavier persons. In such embodiments, the amount of weight loss may be characterized as a different metric, such as the number of kg lost, or a percentage of change in BMI, or may rely on different calculations of BMI, such as via the cubed method (i.e., a Ponderal index), or via a modified formulae such as:

$$1.3 * weight ( kg ) / height ( m ) \hat{} 2.5 = 5734 * weight ( lb ) / height ( in ) \hat{} 2.5 . \qquad ( 1 )$$

The time period, in a manner similar to the selected amount of weight loss, may be a default value (e.g., one year), or may be expressly indicated in the request. Illustrative time periods may comprise one month, three months, six months, one year, two years, etc.

The likelihood 330 is determined by feeding metrics acquired for the patient 320 into the predictive model 254 in order to receive a prediction 328 (e.g., a specific likelihood 330 that the patient 320 will lose a specific amount of weight within the time period). For example, data for each metric may be supplied as an argument to a function calling the predictive model 254. The likelihood 330 may be reported as a numeric score, such as a value between zero and one, a value between zero and one hundred, etc. Alternatively, the likelihood 330 may comprise a classification or categorization, such as “low,” “moderate”, or “high”. In many embodiments, separate predictive models 254 are trained for separate time periods. Hence, information in a request relating to time period may impact a selection of a predictive model 254 performed by controller 232.

Step 312 comprises controller 232 comparing the likelihood 330 to a threshold 332. The threshold 332 may comprise, for example, a percent likelihood, such as sixty percent, seventy-five percent, ninety percent, etc. Comparing the likelihood 330 to the threshold 332 may comprise converting the threshold 332 and the likelihood 330 to common units, and then determining if the likelihood 330 is equal to or higher than the threshold 332.

In an event the likelihood 330 is below the threshold 332, processing continues to step 316. Step 316 comprises controller 232 recommending that the semaglutide dosage 324 not be prescribed to the patient 320. This may comprise providing a recommendation 340 in the form of a report or notification transmitted to provider client 210 for display.

In an event that the likelihood 330 is above the threshold 332, processing continues to step 314. Step 314 comprises recommending that the semaglutide dosage 324 be prescribed to the patient 320. This may comprise providing a recommendation 340 in the form of a report or notification transmitted to provider client 210 for display. Based on this information, a healthcare provider may administer and/or prescribe the semaglutide 322 to the patient 320.

Method 300 provides a notable benefit over prior techniques, because it provides healthcare providers with realistic, data-driven, and bespoke analyses of semaglutide effectiveness for individual patients. This ensures that patients continue to receive not just possible treatments for weight loss, but the best possible treatments available to them for weight loss.

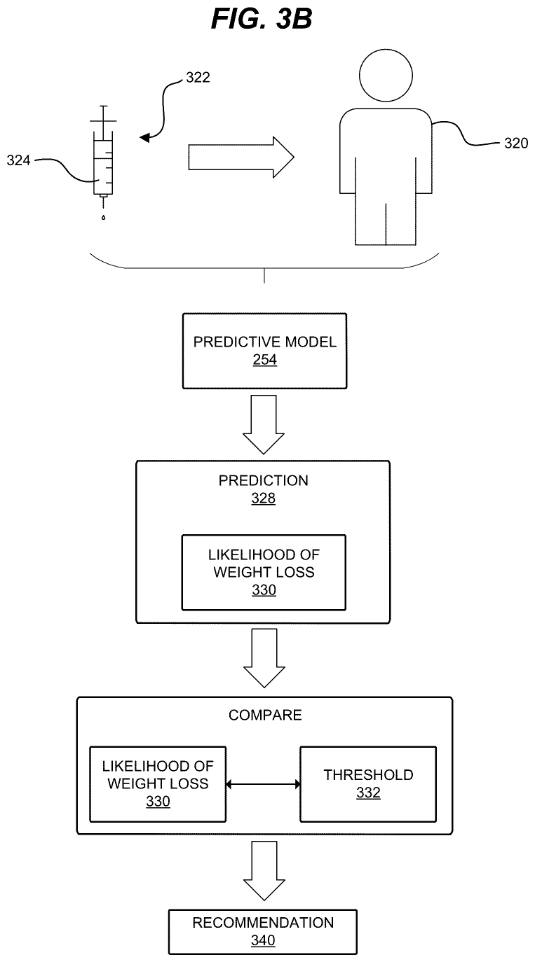

FIG. 4A is a flowchart depicting a method 400 for identifying efficacious lower dosages of semaglutide 322 using population metrics in an illustrative embodiment. Method 400 may be performed, for example, in the event that the likelihood 330 of weight loss for a dosage 324 in method 300 achieving a desired level of weight loss is above the threshold 332, in order to determine whether a lower dosage would also be effective. This has the technical benefit of reducing semaglutide-related side effects experienced by the patient 320 during the prescription period, which in turn is likely to increase the chance of the patient 320 continuing to use semaglutide 322 for the entire prescription period. It has the additional benefit of increasing patient comfort. Furthermore, method 400 may be performed iteratively, in order to progressively inspect the impact of each of multiple downward steps of dosage. FIG. 4B illustrates administration of semaglutide 322 in an illustrative embodiment.

Step 402 comprises controller 232 identifying a lower dosage 424 of semaglutide 322 (see FIG. 4B). For example, controller 232 may have multiple dosages indicated in memory 224, such as 0.5, 1.0, 1.7, 2.0 and 2.4 mg/wk. In many cases, 2.4 mg/wk may comprise the default dosage, and controller 232 may select the next-highest dosage below the dosage that was previously analyzed.

Step 404 comprises controller 232 operating the predictive model 254 to predict a likelihood 430 of the lower dosage 424 accomplishing the selected amount of weight loss for the patient 320 during the time period. The likelihood 430 is determined by feeding metrics acquired for the patient 320 into the predictive model 254 in order to receive a prediction 428 (e.g., a specific likelihood 430 that the patient 320 will lose a specific amount of weight within the time period). For example, data for each metric may be supplied as an argument to a function calling the predictive model 254. This may be performed in a similar manner to step 310 of method 300. However, in some embodiments, a different predictive model 254 tuned to the lower dosage may be used.

Step 406 comprises controller 232 comparing the likelihood 430 for the lower dosage 424 to the threshold 332. This may be performed in a similar manner to step 312 of method 300.

In an event the likelihood 430 for the lower dosage 424 is below the threshold 332, processing continues to step 410. Step 410 comprises recommending that the lower dosage 424 not be prescribed to the patient 320. For example, this may comprise annotating a recommendation 340 for the higher dosage (e.g., the semaglutide dosage 324 of FIG. 3B) with a note that a lower dosage 424 is not expected to achieve the selected amount of weight loss within the period.

In an event the likelihood 430 for the lower dosage 424 is above the threshold 332, processing continues to step 408. Step 408 comprises recommending that the lower dosage 424 be prescribed to the patient 320. This recommendation 340 may be provided in addition to the recommendation 340 for the previously-considered dosage, because both dosages are expected to achieve the desired weight loss. However, the recommendation 340 for the lower dosage 424 may be accompanied by explanatory content indicating that side effects and/or patient expense may be reduced by using the lower dosage 424.

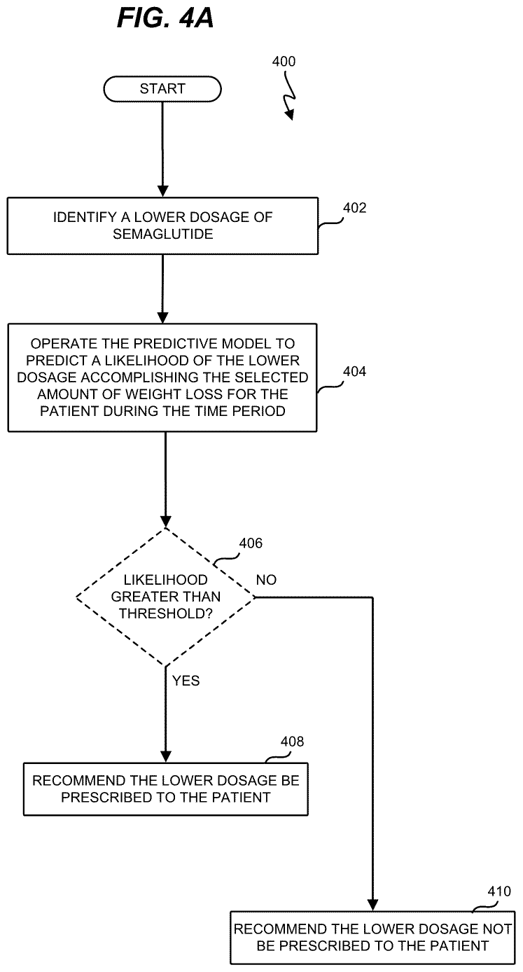

FIG. 5 is a flowchart depicting a method 500 for selecting demographic-specific models for predicting semaglutide effectiveness in an illustrative embodiment. Use of demographic-specific models (e.g., models that have been trained using EHR data within specific demographics matched to the patient) may yield more precise results, in environments where the size of the training data used for the model remains notable (e.g., tens or hundreds of thousands of patients).

Step 502 comprises controller 232 training multiple predictive models 254, where each of the predictive models 254 is trained using metrics specific to one or more demographics in categories selected from the group consisting of: sex, ancestry, age, and Body Mass Index (BMI). This may be performed in a similar manner to step 306 of method 300, using a separate population belonging to each demographic group as training data for each separate predictive model 254.

In alternate embodiments, training data may not be of sufficient size to create individual models trained only on data from specific demographic groups. In such embodiments, controller 232 may alternatively set dynamic, varying thresholds 332 while continuing to use the same predictive model 254. For example, a threshold 332 for each group may be set to a top quintile or quartile value found for the demographic corresponding to the patient.

Step 504 comprises controller 232 selecting and operating one of the predictive models 254 based on demographics of the patient 320. This may be performed by identifying a predictive model 254 that was trained on each demographic that the patient 320 belongs to, or at least one demographic that the patient 320 belongs to. In further embodiments, demographics are ranked based on priority (e.g., sex, followed by age, followed by ancestry), and a predictive model 254 trained on the highest-priority demographic is utilized.

In alternate embodiments where the threshold 320 is dynamically varied for a single model, controller 232 may select a threshold 320 based on similar criteria, and operate the single predictive model 254 while using a dynamic threshold 320.

These techniques provide a technical benefit by helping to enhance precision-insights for patients by utilizing data specific to demographic groups for those patients.

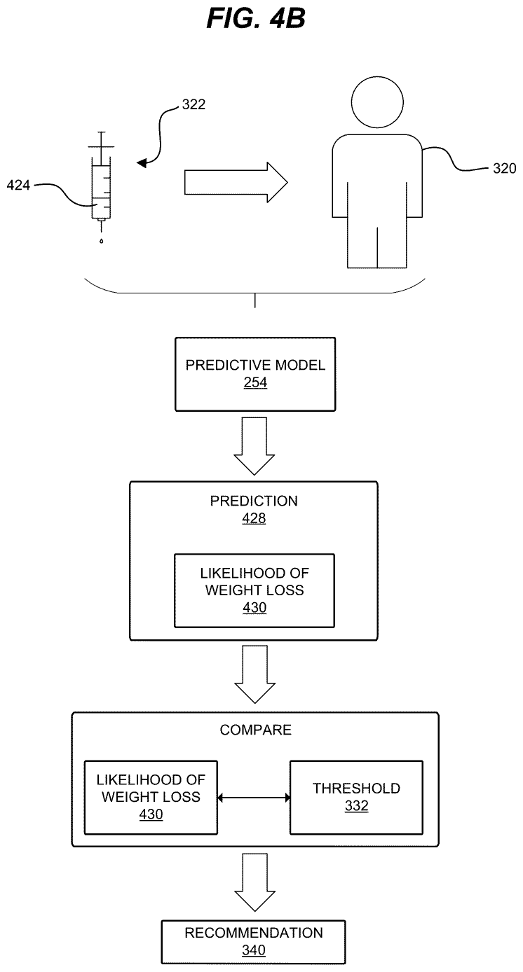

FIG. 6 is a flowchart depicting a method 600 selectively administering semaglutide 322 to a patient 320 in an illustrative embodiment. Method 600 may be performed, for example, by a healthcare provider that is selecting a method of treatment for a patient 320.

Step 602 comprises identifying a patient 320. This may comprise selecting a patient identifier for a patient 320 visiting the healthcare provider, via provider client 210. In further embodiments, this comprises entering a name for the patient 320, or selecting the patient 320 from a list. In many embodiments, provider client 210 will have access to EHR data for the patient 320, which may mirror or supplement EHR data 252.

Step 604 comprises selecting a dosage 324 of semaglutide 322 for the patient 320. This may be performed by the healthcare provider operating provider client 210 to select a dosage 324, or by the healthcare provider remaining silent and therefore provider client 210 selecting a default dosage, such as 2.4 mg/wk.

Step 606 comprises operating a predictive model 254 trained upon health data for a population using metrics of sex, sleep apnea, hypertension, and prescription history to predict a likelihood 330 of the semaglutide dosage 324 accomplishing a loss of at least ten percent body mass for the patient 320 during a time period of one year.

Operating the predictive model 254 may comprise transmitting a message to health server 220 via provider client 210, requesting a prediction 428 of semaglutide effectiveness for the patient 320. In further embodiments, operating the predictive model 254 may comprise controller 232 actively making a prediction 428 for the patient 320 by using the predictive model 254, and transmitting a response that includes a likelihood 330 of achieving a selected amount of weight loss for the patient 320 back to provider client 210.

Step 608 comprises comparing the likelihood 330 to a threshold 332. This may be performed in a similar manner to step 312 of method 300. In an event the likelihood 330 is below the threshold 332, processing continues to step 612. Step 612 comprises preventing administration of the dosage 324 to the patient 320. This may comprise, for example, making a note in EHR data for the patient 320 that the patient 320 would not benefit from, and should not be prescribed, semaglutide 322 for purposes of weight loss.

In an event the likelihood 330 is above a threshold 332, processing continues to step 610. Step 610 comprises administering the dosage 324 to the patient 320. This may comprise injecting the dosage 324 of semaglutide 322 into the patient 320, or writing a prescription that permits the patient 320 to self-inject the dosage 324.

Method 600 provides a technical benefit by providing a precise, specific technique for proceeding with or foregoing administering semaglutide 322 to patients 320 for weight loss purposes.

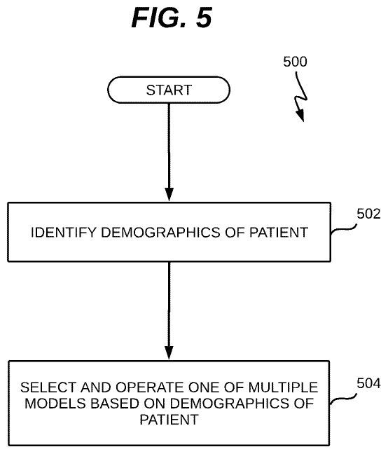

FIG. 7 is a table 700 that summarizes sequencing data for one or more genes for individuals in an illustrative embodiment. For example, table 700 may be one of many data structures stored in health server 220. In this embodiment, table 700 includes an entry 710 for each of multiple patients. Each entry 710 includes a unique identifier (e.g., LSID) for the corresponding patient, as well as an indication of the gene that the sequencing data relates to. The portion of the genome that has been sequenced may comprise whole genome data, whole exome data, array data, data for a specific gene or portion of a gene, etc. Table 700 also indicates a format of the sequencing data. Table 700 may be generated based on, or with reference to, sequences that have been alignment-enhanced via the processes discussed above.

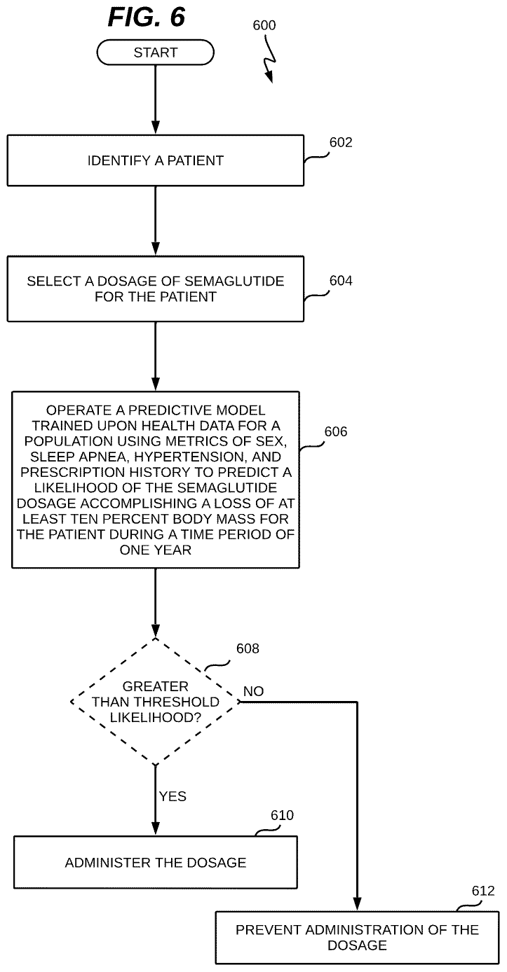

FIG. 8 is a table 800 that summarizes variant data for individuals in an illustrative embodiment. In this embodiment, each entry 810 in table 800 reports a location (e.g., chromosomal coordinate) for each genetic variant, together with flags indicating whether the variant is a Loss of Function (LoF) variant or a coding variant. Table 800 further includes a VCF reference, which refers to the location and/or identifier of a VCF file that indicates the presence of the variant. The VCF file may be generated using data from the alignment enhancement processes discussed above. For example, alignment-enhanced data in a BAM, SAM, or CRAM file may include data used to generate the VCF file. Table 800 may be utilized by controller 232 of health server 220, in order to rapidly select and report diagnostic and treatment thresholds for a patient. Table 800 may be generated based on, or with reference to, sequences that have been alignment-enhanced via the processes discussed above.

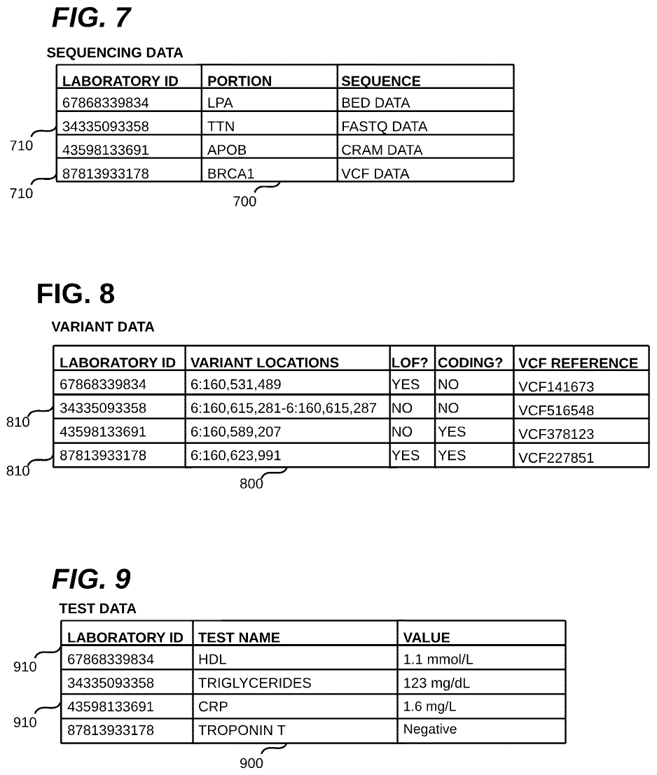

FIG. 9 is a table 900 that summarizes biomarker test data for individuals in an illustrative embodiment. Specifically, table 900 summarizes test data pertaining to predetermined diseases for each of multiple patients in an illustrative embodiment. Each entry 910 in table 900 indicates an anonymized laboratory ID for a patient, a corresponding test name, and a corresponding value. Table 900 may be created, for example, based on EHR data retrieved for patients. Laboratory IDs may be associated with EHR identifiers at health server 220 or provider client 210, in order to enable access to both health data and genomics data for a patient. Table 900 may be used to enhance or provide context for genetic insights determined based sequences that have been alignment-enhanced via the processes discussed above.

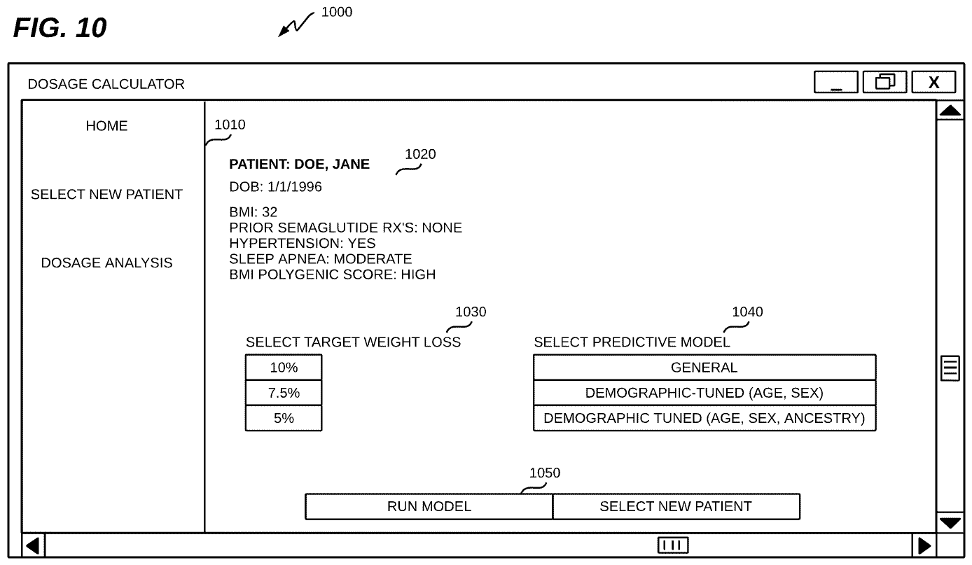

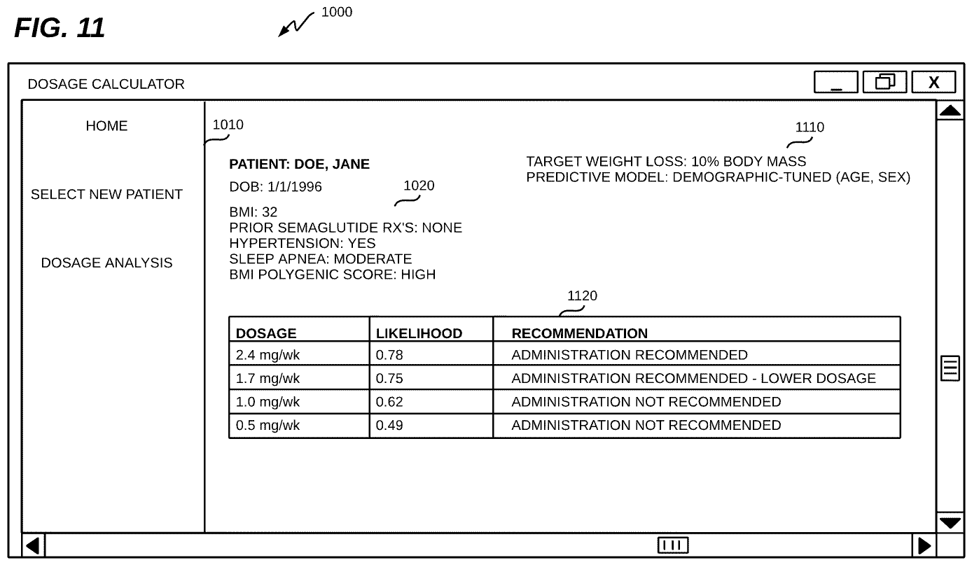

FIGS. 10-11 depict Graphical User Interfaces (GUIs) that facilitate the analysis of semaglutide effectiveness for patients in illustrative embodiments. These GUIs may be presented, for example, via a browser window or other portion of a screen of one or more provider clients 210.

FIG. 10 depicts a GUI 1000 that facilitates ex-ante estimates of semaglutide effectiveness in an illustrative embodiment. For example, GUI 1000 may be implemented via provider client 210 in order to facilitate user operations pertaining to predicting effectiveness of semaglutide 322 in specific patients.

In this embodiment, GUI 1000 includes a menu portion 1010. Menu portion 1010 provides access to multiple windows/pages at GUI 1000. These portions include a home page, which provides access to prior-generated predictions for subjects, a selection page which permits the user to select a patient 320 (e.g., via a unique ID for the patient used by the healthcare provider, such as a Medical Record Number (MRN) or similar), and a dosage analysis page providing details relating to the predicted effectiveness of semaglutide 322 for a specific patient 320.

As depicted, GUI 1000 displays a dosage analysis page, which includes patient information and metrics portion 1020, as well as an interactive element 1030 for target weight loss selection, an interactive element 1040 for selecting a predictive model 254, and an interactive element 1050 for triggering operation of the selected predictive model 254 based on information for the patient 320 (e.g., by transmitting a request to health server 220).

FIG. 11 depicts the GUI 1000 of FIG. 10, this time displaying an updated dosage analysis page, after analysis has been completed. In FIG. 11, an analysis summary portion 1110 indicates how the prediction of semaglutide effectiveness was made. Meanwhile, details portion 1120 reports one or more dosages, likelihoods, and/or recommendations pertaining thereto. Based on these recommendations, a healthcare practitioner may initiate, alter, discontinue, or prevent administration of semaglutide to the patient under consideration.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein, such as health server 220, to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by an entity. Cloud computing relies on the sharing of resources, and generally includes on-demand self-service, broad network access, resource pooling, rapid elasticity, and measured service.

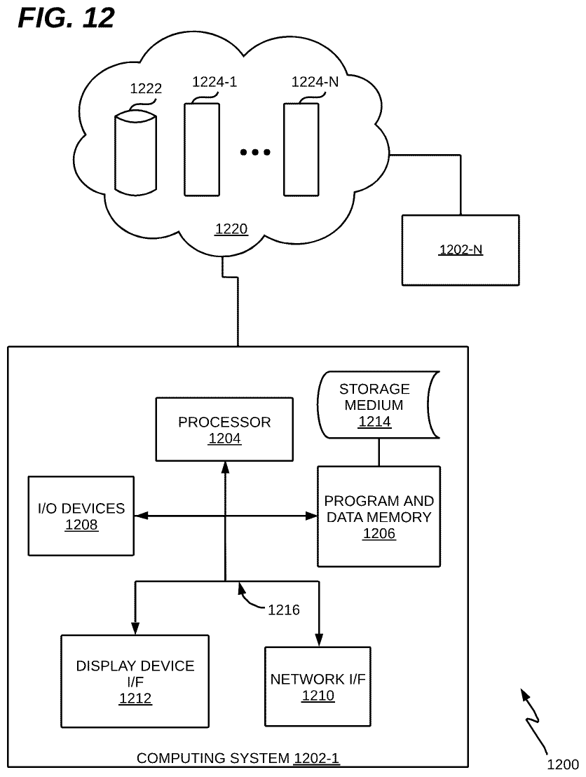

FIG. 12 depicts one illustrative cloud computing system 1200 operable to perform the above operations by executing programmed instructions tangibly embodied on one or more computer readable storage mediums. The cloud computing system 1200 generally includes the use of a network of remote servers hosted on the internet to store, manage, and process data, rather than a local server or a personal computer (e.g., in the computing systems 1202-1, 1202-N). Cloud computing enables users to use infrastructure and applications via the internet, without installing and maintaining them on-premises. In this regard, the cloud computing network 1220 may include virtualized information technology (IT) infrastructure (e.g., servers 1224-1-1224-N, the data storage module 1222, operating system software, networking, and other infrastructure) that is abstracted so that the infrastructure can be pooled and/or divided irrespective of physical hardware boundaries. In some embodiments, the cloud computing network 1220 can provide users with services in the form of building blocks that can be used to create and deploy various types of applications in the cloud on a metered basis.

Various components of the cloud computing system 1200 may be operable to implement the above operations in their entirety or contribute to the operations in part. For example, a computing system 1202-1 may be used to perform analysis of gene sequencing data, and then store that analysis along with the gene sequencing data in a data storage module 1222 (e.g., a database) of a cloud computing network 1220. Various computer servers 1224-1-1224-N of the cloud computing network 1220 may be used to operate on the gene sequencing data and/or transfer the gene sequencing analysis and/or the gene sequencing data to another computing system 1202-N.

Some embodiments disclosed herein may utilize instructions (e.g., code/software) accessible via a computer-readable storage medium for use by various components in the cloud computing system 1200 to implement all or parts of the various operations disclosed hereinabove. Examples of such components include the computing systems 1202-1, 1202-N.

Exemplary components of the computing systems 1202-1, 1202-N may include at least one processor 1204, a computer readable storage medium 1214, program and data memory 1206, input/output (I/O) devices 1208, a display device interface 1212, and a network interface 1210. For the purposes of this description, the computer readable storage medium 1214 comprises any physical media that is capable of storing a program for use by the computing system 1202. For example, the computer-readable storage medium 1214 may be an electronic, magnetic, optical, electromagnetic, infrared, semiconductor device, or other non-transitory medium. Examples of the computer-readable storage medium 1214 include a solid-state memory, a magnetic tape, a removable computer diskette, a random access memory (RAM), a read-only memory (ROM), a rigid magnetic disk, and an optical disk. Some examples of optical disks include Compact Disk-Read Only Memory (CD-ROM), Compact Disk-Read/Write (CD-R/W), Digital Versatile Disc (DVD), and Blu-Ray Disc.

The processor 1204 is coupled to the program and data memory 1206 through a system bus 1216. The program and data memory 1206 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 1208 (including but not limited to keyboards, displays, touchscreens, microphones, pointing devices, etc.) may be coupled either directly or through intervening I/O controllers. Network adapter interfaces 1210 may also be integrated with the system to enable the computing system 1202 to become coupled to other computing systems or storage devices through intervening private or public networks. The network adapter interfaces 1210 may be implemented as modems, cable modems, Small Computer System Interface (SCSI) devices, Fibre Channel devices, Ethernet cards, wireless adapters, etc. Display device interface 1212 may be integrated with the system to interface to one or more display devices, such as screens for presentation of data generated by the processor 1204.

What is claimed is:

1. A system for analyzing dosage effectiveness, the system comprising:
an interface configured to receive health data for a population of patients; and
a controller configured to extract metrics for sex, sleep apnea, hypertension, and prescription history from the health data, and to train a predictive model based on the metrics;
the controller further configured to identify a patient and a semaglutide dosage, and to operate the predictive model to predict a likelihood of the semaglutide dosage accomplishing a selected amount of weight loss for the patient during a time period;
in an event the likelihood for the semaglutide dosage is below a threshold, the controller is further configured to recommend that the semaglutide dosage not be prescribed to the patient;
in an event the likelihood for the semaglutide dosage is above the threshold, the controller is further configured to recommend that the semaglutide dosage be prescribed to the patient.

2. The system of claim 1 wherein:
the controller is further configured, in the event that the likelihood for the semaglutide dosage is above the threshold, to identify a lower dosage of semaglutide, and to operate the predictive model to predict a likelihood of the lower dosage accomplishing the selected amount of weight loss for the patient during the time period,
in an event the likelihood for the lower dosage is below the threshold, the controller is further configured to recommend that the lower dosage not be prescribed to the patient; and
in an event the likelihood for the lower dosage is above the threshold, the controller is further configured to recommend that the lower dosage be prescribed to the patient.

3. The system of claim 1 wherein:
the metrics further comprise a polygenic score for Body Mass Index (BMI), concurrent use of a non-Glucagon-like peptide-1 (GLP-1) weight loss drug with semaglutide, and previous use of another GLP-1 weight loss drug within a year immediately prior to prescription of semaglutide.

4. The system of claim 1 wherein:
the selected amount of weight loss is at least ten percent of body weight, and the threshold is between fifty and ninety-nine percent.

5. The system of claim 1 wherein:
the controller is further configured to train multiple predictive models, each of the predictive models trained using metrics specific to one or more demographics in categories selected from the group consisting of: sex, ancestry, age, and Body Mass Index (BMI); and
the controller is further configured to select one of the predictive models based on demographics of the patient.

6. The system of claim 1 wherein:
the controller is further configured to train the predictive model using logistic regression.

7. The system of claim 1 wherein:
the controller is further configured to exclude metrics for patients of the population that have type two diabetes, prior to training the predictive model.

8. A method for analyzing dosage effectiveness, the method comprising:
receiving health data for a population of patients;
extracting metrics for sex, sleep apnea, hypertension, and prescription history from the health data;
training a predictive model based on the metrics;
identifying a patient and a semaglutide dosage;
operating the predictive model to predict a likelihood of the semaglutide dosage accomplishing a selected amount of weight loss for the patient during a time period;
in an event the likelihood for the semaglutide dosage is below a threshold, recommending that the semaglutide dosage not be prescribed to the patient; and
in an event the likelihood for the semaglutide dosage is above the threshold, recommending that the semaglutide dosage be prescribed to the patient.

9. The method of claim 8 further comprising:
in the event that the likelihood for the semaglutide dosage is above the threshold:
identifying a lower dosage of semaglutide;
operating the predictive model to predict a likelihood of the lower dosage accomplishing the selected amount of weight loss for the patient during the time period;
in an event the likelihood for the lower dosage is below the threshold, recommending that the lower dosage not be prescribed to the patient; and
in an event the likelihood for the lower dosage is above the threshold, recommending that the lower semaglutide dosage be prescribed to the patient.

10. The method of claim 8 wherein:
the metrics further comprise a polygenic score for Body Mass Index (BMI), concurrent use of a non-Glucagon-like peptide-1 (GLP-1) weight loss drug with semaglutide, and previous use of another GLP-1 weight loss drug within a year immediately prior to prescription of semaglutide.

11. The method of claim 8 wherein:
the selected amount of weight loss is at least ten percent of body weight, and the threshold is between fifty and ninety-nine percent.

12. The method of claim 8 further comprising:
training multiple predictive models, each of the predictive models trained using metrics specific to one or more demographics in categories selected from the group consisting of: sex, ancestry, age, and Body Mass Index (BMI); and
selecting one of the predictive models based on demographics of the patient.

13. The method of claim 8 wherein:
training the predictive model comprises using logistic regression.

14. The method of claim 8 further comprising:
excluding metrics for patients of the population that have type two diabetes, prior to training the predictive model.

15. A non-transitory computer readable medium embodying programmed instructions which, when executed by a processor, are operable for performing a method for analyzing dosage effectiveness, the method comprising:
receiving health data for a population of patients;
extracting metrics for sex, sleep apnea, hypertension, and prescription history from the health data;
training a predictive model based on the metrics;
identifying a patient and a semaglutide dosage;
operating the predictive model to predict a likelihood of the semaglutide dosage accomplishing a selected amount of weight loss for the patient during a time period;
in an event the likelihood for the semaglutide dosage is below a threshold, recommending that the semaglutide dosage not be prescribed to the patient; and
in an event the likelihood for the semaglutide dosage is above the threshold, recommending that the semaglutide dosage be prescribed to the patient.

16. A method for administering semaglutide, the method comprising:
identifying a patient;
selecting a dosage of semaglutide for the patient;
operating a predictive model trained upon health data for a population using metrics of sex, sleep apnea, hypertension, and prescription history to predict a likelihood of the dosage accomplishing a loss of at least ten percent body mass for the patient during a time period of one year;
in an event the likelihood is below a threshold, preventing administration of the dosage to the patient; and
in an event the likelihood is above the threshold, administering the dosage to the patient.

17. The method of claim 16 wherein:
the threshold is between fifty and ninety-nine percent.

18. The method of claim 16 wherein:
operating the predictive model comprises operating a logistic regression model.

19. The method of claim 16 wherein:
the predictive model is further trained upon a polygenic score for Body Mass Index (BMI), concurrent use of a non-Glucagon-like peptide-1 (GLP-1) weight loss drug with semaglutide, and previous use of another GLP-1 weight loss drug within a year immediately prior to prescription of semaglutide.

20. The method of claim 16 wherein:
the population comprises a population of patients, excluding patients having type two diabetes.
